(19) United States
(12) Patent Application Publication
(10) Pub. No.: US 2018/0268933 A1
(43) Pub. Date: Sep. 20, 2018

(54) HEALTHCARE COST ALIGNMENT SYSTEM

(71) Applicants: Adam Laurin, Sussex, WI (US); Keith Kieffer, Hartland, WI (US)

(72) Inventors: Adam Laurin, Sussex, WI (US); Keith Kieffer, Hartland, WI (US)

(73) Assignee: Milliman, Inc., Brookfield, WI (US)

(21) Appl. No.: 15/918,801

(22) Filed: Mar. 12, 2018

Related U.S. Application Data
(60) Provisional application No. 62/471,147, filed on Mar. 14, 2017.

(51) Int. Cl. G16H 40/20; G06Q 40/08

(52) U.S. Cl. CPC G16H 40/20; G06Q 40/08

(57) ABSTRACT

A method of aligning costs including providing a networked computer server configured to receive, via the network, data for each of a plurality of members in the healthcare system, aligning a portion of the plurality of members with a healthcare provider using a member alignment algorithm, and calculating a cost target for total healthcare costs for a period of time using a cost target algorithm. The cost target is based on historical medical costs. The method includes determining a contribution margin with a fixed cost and a profit margin for the healthcare provider, determining variable costs for one or more healthcare services, and calculating fixed cost payments to the healthcare provider on a per-member-basis. The method requires receiving claim data, via the network, for claims made by the plurality of members, and calculating claim payments, based on the variable costs, on a per-member-basis.

### CROSS-REFERENCE TO RELATED PATENT APPLICATION

This patent application claims the benefit of U.S. Provisional Patent Application No. 62/471,147, filed Mar. 14, 2017, the entire teachings and disclosure of which are incorporated herein by reference thereto.

### FIELD OF THE INVENTION

This invention generally relates to systems and methods for managing healthcare costs.

### BACKGROUND OF THE INVENTION

Employers are faced with increasing healthcare costs for the health benefits they provide to their employees. Providers are In the best position to improve the efficiency of care and lower costs, however, under traditional fee-for-service payment structures, lower utilization also means lower profitability for the healthcare providers. One of the strategies to combat increasing costs has been a movement from traditional fee for service payments to value based care reimbursement structures which provide incentives for healthcare providers to manage the cost of the care they provide. However, most current value based reimbursement models do not provide sufficient incentives for providers to reduce utilization in a meaningful manner. The few models which provide sufficient incentives to lower utilization have significant disadvantages for the employers who would benefit from the lower utilization. Embodiments of the present invention achieve both goals of providing incentives to lower overall healthcare expenditures for employers while avoiding the disadvantages of other financial models.

Embodiments of the invention provide a system and method to address some of the issues affecting healthcare costs described above. These and other advantages of the invention, as well as additional inventive features, will be apparent from the description of the invention provided herein.

### BRIEF SUMMARY OF THE INVENTION

In one aspect, embodiments of the invention provide a method of aligning costs in a healthcare system that includes providing a networked computer server configured to receive, via the network, personal and health data for each of a plurality of members in the healthcare system. The method also includes aligning at least a portion of the plurality of members with a healthcare provider using a member alignment algorithm executed by the computer server, and calculating a preliminary cost target for total healthcare costs for a period of time using a cost target algorithm executed by the computer server. The preliminary cost target is based on a baseline of historical medical costs. The method includes determining a contribution margin that includes both a fixed cost and a profit margin for the healthcare provider, determining a variable cost for one or more healthcare services, and calculating fixed cost payments to the healthcare provider on a per-member-basis. The method also calls for receiving claim data, via the network, for one or more claims made by the one or more of the plurality of members, and calculating claim payments on a per-member-basis, the claim payments based on the variable costs associated with the claim for healthcare services.

In a particular embodiment, the method includes tracking all fixed cost payments and all claim payments for the period of time to determine a revised healthcare cost, and comparing the revised healthcare cost to the preliminary cost target. The method may also include calculating a risk sharing component where any cost saving realized when the revised healthcare cost is less than the preliminary cost target, or any cost overage realized when the revised healthcare cost is greater than the preliminary cost target, is divided between the healthcare provider and a customer of the healthcare provider. In certain embodiments, the method requires configuring the computer server to allow a user to adjust how the risk sharing component is divided.

In a further embodiment, the method includes calculating an adjusted risk score for a member using a risk adjustment algorithm executed on the computer server. The adjusted risk score indicates a healthcare cost risk for the member as compared to a sample population. The method may call for calculating an adjusted cost target based on the adjusted risk score for each of the plurality of members. The method may also call for determining a revised healthcare cost comprises excluding some claim payments to get an adjusted revised healthcare cost. In more particular embodiments, the method includes excluding some claim payments comprises excluding some claim payments above a certain threshold amount.

In some embodiments, the method includes calculating a preliminary cost target for total healthcare costs using a cost target algorithm comprises excluding some healthcare costs from the historical baseline cost to get an adjusted baseline cost, and trending the adjusted baseline cost forward to cover a current period of time. The method may further include excluding some healthcare costs comprises excluding some healthcare costs above a certain threshold amount. In a further embodiment, the method comprises pooling members of two or more employer's before calculating the preliminary cost target for the pooled members. In certain embodiments, the method requires pooling of an employer's members when the member population of the employer is less than a threshold level.

In another aspect, embodiments of the invention provide a healthcare cost alignment system that includes a computer server configured to communicate, via a communications network, with one or more client computers in order to send and receive healthcare information over the communications network. The computer server configured to receive personal and healthcare data for each of a plurality of members in the healthcare system, align each of the plurality of members with a healthcare provider based on a set of criteria for member alignment, and determine a preliminary cost target for total healthcare costs based on historical data and the personal and healthcare data. The computer server also determines a variable cost for one or more healthcare services. The computer server is configured to receive, over the network, claim data for one or more claims made by one or more of the plurality of members, and calculate a claim payment amount for each of the one or more claims based on variable costs associated with the claim at issue.

In a particular embodiment, the computer server is further configured to track all fixed cost payments and all claim payments for the period of time to determine a revised healthcare cost, and compare the revised healthcare cost to the preliminary cost target. The computer server may be further configured to calculate a risk sharing component where any cost saving realized when the revised healthcare cost is less than the preliminary cost target, or any cost overage realized when the revised healthcare cost is greater than the preliminary cost target, is divided between the healthcare provider and a customer of the healthcare provider.

In some embodiments, the computer server is configured to allow a user to adjust how the risk sharing component is divided. In other embodiments, the computer server is further configured to calculate an adjusted risk score for a member using a risk adjustment algorithm executed on the computer server. The adjusted risk score indicates a healthcare cost risk for the member as compared to a sample population.

The computer server may be configured to calculate an adjusted cost target based on the adjusted risk score for each of the plurality of members. Further, the computer server may be configured to determine a revised healthcare cost that excludes some claim payments in order to get an adjusted revised healthcare cost. In certain embodiments, the computer server is further configured to exclude some claim payments above a certain threshold amount and/or aligned members who meet certain conditions. The computer server may also be configured to exclude some healthcare costs from the historical baseline cost to get an adjusted baseline cost, and configured to trend the adjusted baseline cost forward to cover a current period of time. Also, the computer server may be further configured to exclude some healthcare costs above a certain threshold amount.

In a particular embodiment, the computer server is further configured to pool members of two or more employers before calculating the preliminary cost target for the pooled members. In a more particular embodiment, the computer server automatically pools the members of any employer with less than a threshold number of members.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a schematic diagram illustrating a healthcare cost alignment system, according to an embodiment of the invention;

FIG. 2 is a flowchart illustrating a method for alignment costs within a healthcare system, in accordance with an embodiment of the invention;

FIG. 3 is a flowchart showing how the method of FIG. 2 can be modified to incorporate adjusted risk scores in the generation of cost targets, according to an embodiment of the invention; and

FIG. 4 is a flowchart showing how the method of FIG. 2 can be modified to incorporate pooling of employer populations, according to an embodiment of the invention.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Embodiments of the invention described herein address many of the shortcomings described above with respect to conventional healthcare systems. The model healthcare system of the present invention is intended to provide stable platform for the members of the health system, and to provide incentives to the healthcare provider for long- and short-term cost management. Further, this model can be adopted without the need for wholesale changes to the current healthcare system. As will be shown below, the health care system of the present invention provides incentives for healthcare providers to lower costs by dis-incentivizing the providing of unnecessary or redundant medical procedures as a means for increasing profit. Because profits of the healthcare providers are not increased by each new procedure, there is some incentive to provide only those services and procedures needed to produce a positive outcome.

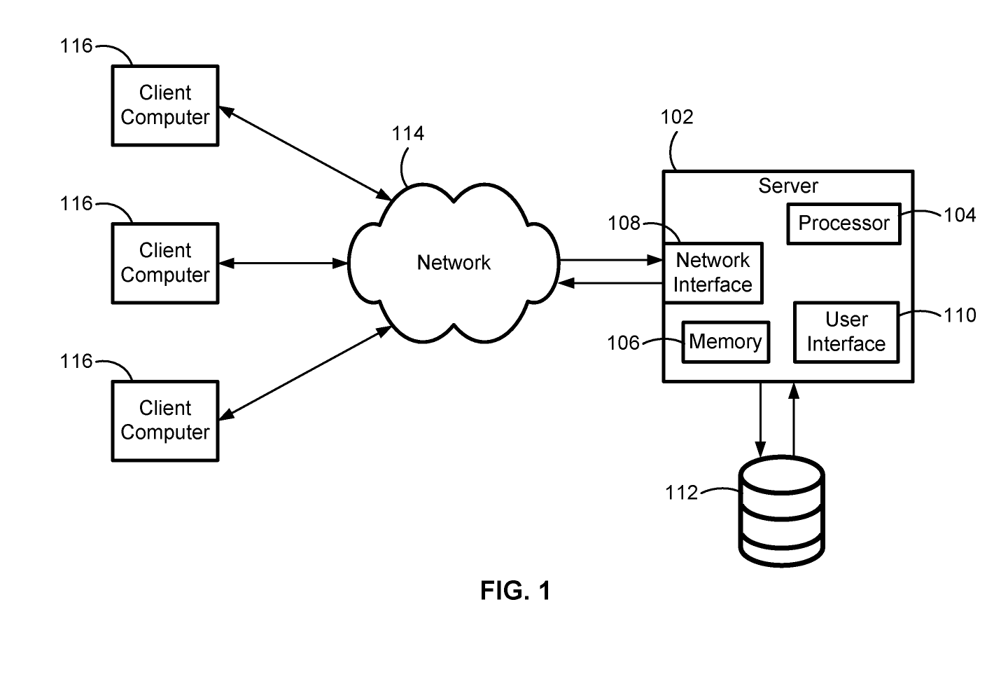

FIG. 1 shows a schematic diagram illustrating a healthcare cost alignment system 100, according to an embodiment of the invention. The system 100 includes a computer server 102 having a processor 104, computer memory 106, and a network interface 108. The server 102 has a user interface 110, which may include, among other things, a display and input/output means for the user. The server further includes a database 112 for storage of healthcare information, including costs associated with the provision of healthcare.

As shown in FIG. 1, the server 102 for the healthcare cost alignment system 100 is coupled to a communications network 114, such as the internet or a private intranet, to facilitate two-way communication between the server 102 and client computers 116. In a particular embodiment of the invention, it is envisioned that the client computers 116, most typically in communication with the server 102, will belong to healthcare providers; employers whose employees are members of a healthcare system supported by the healthcare cost alignment system 100; health care administrators such as insurance companies that perform healthcare administrative functions, such as claim processing, for the employer on behalf the employees/members.

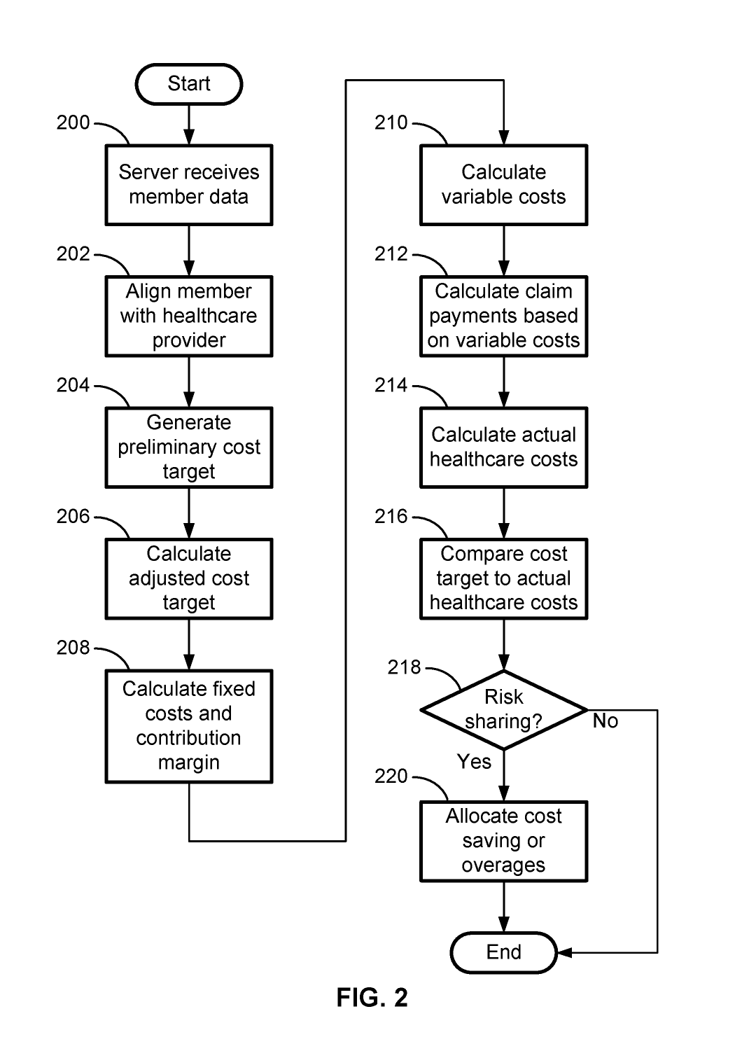

FIG. 2 is a flowchart illustrating a method for alignment costs within a healthcare system, in accordance with an embodiment of the invention. In an initial step, the server 102 receives information, including personal and health-related information, for each of the members participating in the cost alignment process 200. This data may be stored in the database 112 (shown in FIG. 1). In certain embodiments, the server 102 is programmed to perform one or more quality validations on all member data to verify that the data provided is accurate and up-to-date. The server 102 may be further programmed to flag any data found deficient, and to automatically request corrected data from the employer or health care administrator.

Once the member data is collected, some or all of the members are aligned with a health care provider 202. In particular embodiments, the server 102 is programmed to execute a member alignment algorithm which assigns members to a health care provider based on multiple criteria that can be set or modified by the user. Potential criteria include, for example, a member's treatment history, demographic and morbidity factors, or geographical location. Further, the server 102 may be programmed to receive, via the network, a request for, or selection of, a particular health care provider

Using the healthcare history obtained from its members, embodiments of the healthcare cost alignment system 100 are configured to generate a preliminary cost target 204 representing the total health care costs over a given period of time for an organization, such as the aforementioned employer. In a particular embodiment, the cost preliminary target is generated using historical healthcare cost data for each of the plurality of members in the healthcare system. In an embodiment of the invention, a cost target algorithm, executed on the server 102, analyzes the personal and health data of the members, along with their respective medical histories to develop a cost trend that can be applied to the historical healthcare costs to generate the preliminary cost target 204. In some embodiments of the invention, the server is programmed to calculate a risk score for each member of the healthcare system. The risk score indicates the level of healthcare risk associated with an individual as compared, for example, to that of the general population or of a sample population. An individual risk score is based on the personal and health data of a corresponding member. Collectively, all of the risk scores are used by the server 102 to calculate the preliminary cost target 204 for the health care system.

In certain embodiments, the preliminary cost target may be adjusted 206 by removing certain unusual healthcare costs or members who meet certain criteria from the preliminary cost target calculation. For example, healthcare costs over a certain threshold amount may be removed as being one-time occurrences which are not consistent with the principals of the cost sharing agreement between the employer and health system. The server 102 may be programmed to remove healthcare costs based solely on the amount, or may be programmed to take the reason for the expenditure into account before removing the costs from the preliminary cost target calculation. After the identified healthcare costs are removed, the server 102 determines the adjusted cost target 206, for example, on a per-member basis for a given period of time. In practice, the server 102 may be configured to calculate the adjusted cost target 206, for example, on a per-member per-month, per-quarter, or per-year basis by dividing the adjusted cost target, respectively, by the number of member months, member quarters, or member years.

Once the adjusted cost target has been determined, the server 102 uses the data on historical healthcare costs to determine, generally, the portions of those costs that are fixed, and the portions of the costs that are variable. Once the fixed costs are determined, the server 102 is configured to add a profit margin to the health system's fixed cost to yield a contribution margin 208. The contribution margin may also be calculated on a per-member per-month, per-quarter, or per-year basis by dividing the contribution margin, respectively, by the number of member months, member quarters, or member years. The server 102 also determines the variable cost 210 for a variety of medical procedures. As a result, when the server 102 receives claims data for its healthcare system members via the network, claim payments for services rendered by the healthcare provider are automatically calculated and reported based on the determination of variable costs 212 by the server 102.

The effect of the contribution margin, where the healthcare provider receives payments covering fixed cost and profits, is that the healthcare providers have no incentive to provide unnecessary services because the payments for these services, being based on variable costs only, do not increase the profits of the healthcare provider. Thus, the economic interests of the healthcare provider are more aligned with the interests of the member such that only those medical services necessary for a positive medical outcome are provided.

The server 102 is also configured to track the revised healthcare costs for each of its members 214. These healthcare costs are totaled for the same period of time used to determine the preliminary cost target. For example, in certain embodiments, the server 102 is programmed to compare the total revised healthcare costs for a year to an annual cost target 216 for the healthcare system. As in the calculation of the preliminary cost target, the calculation of revised healthcare costs may be adjusted by excluding certain actual healthcare costs. The excluded actual healthcare costs may be those above a certain threshold value, or that meet other predetermined criteria to justify the exclusion.

If the total of revised healthcare costs (or adjusted revised healthcare costs) exceeds the annual cost target, there may be additional healthcare costs to be borne by the employer. On the other hand, if the total of revised healthcare costs exceeds the annual cost target, there may be healthcare cost savings to be realized by the employer. It is also possible that the healthcare provider may share in the additional costs or savings generated under the cost alignment system described above. In embodiments of the invention, the server 102 is programmed to implement the terms of a risk-sharing arrangement 218 if such an arrangement exists.

For example, if there is a risk sharing arrangement 218 between the employer and healthcare provider, the server 102 may be programmed to automatically allocate a portion of the additional cost or savings to the employer and a portion to the healthcare provider. The additional cost or savings may be split evenly between the employer and healthcare provider, or may be the split in any proportion agreed upon by the employer and healthcare provider. In an embodiment, the system user is able to modify the terms (e.g., the allocation percentages) of the risk-sharing arrangement. This allocation may involve a reporting function automatically carried out by the server 102, where the server 102 generates a report detailing the additional healthcare costs or savings allocated to the employer and healthcare provider.

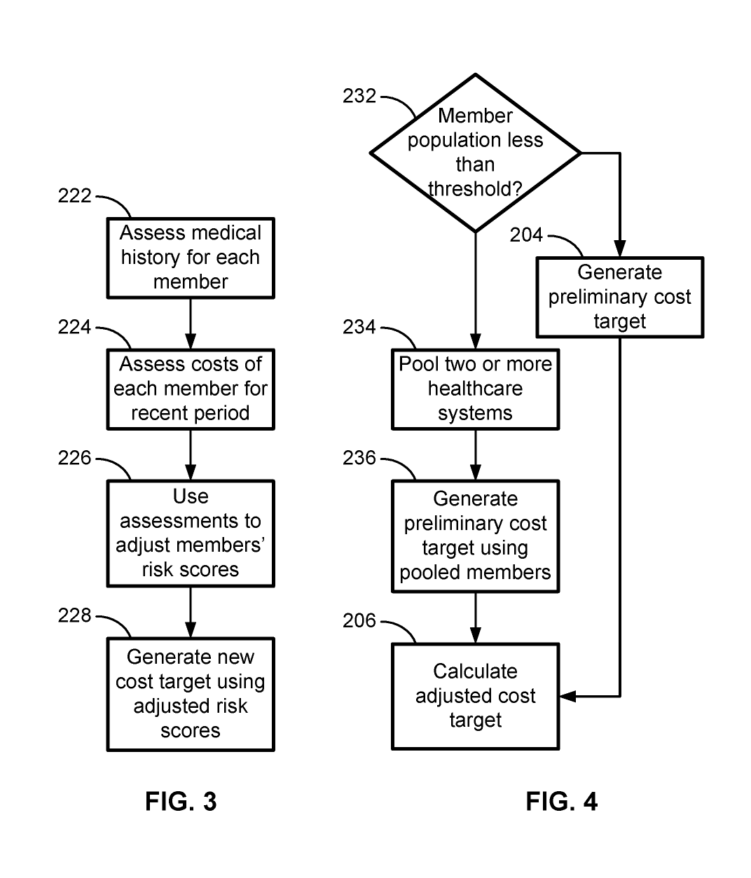

FIG. 3 is a flowchart showing how the method of FIG. 2 can be modified to incorporate adjusted risk scores in the generation of subsequent cost targets, according to an embodiment of the invention. At the end of the time period covered by the preliminary cost target and total of revised healthcare costs, the server 102 may be programmed to perform a risk adjustment for each of its members using a risk adjustment algorithm. In particular embodiments, by analyzing a member's demographic and morbidity factors 222 and the claims history for the most recent time period 224, the server 102 calculates an adjusted risk score for each member of the healthcare system 226. The plurality of adjusted risk scores calculated and stored (e.g., in the database 112) by the server 102 are used to calculate a subsequent health care cost target 228. In certain embodiments, steps 222 through 228 are performed after step 220 shown in FIG. 2.

FIG. 4 is a flowchart showing how the method of FIG. 2 can be modified to incorporate pooling of employer member populations by configuring the server 102 to detect when the member population is below a certain threshold level 232, according to an embodiment of the invention. In some cases, the number of members provided by the employer may be too small to generate a reliable cost target. It is expected that the healthcare costs for individual members will fluctuate from year to year. For example, in one year, a member may have an accident or suffer an illness that requires an expensive surgical procedure, but have no unusual medical expenses for the following ten years. In small populations, these unexpected high-cost healthcare expenses will cause the costs to noticeably fluctuate from year to year. However, in large populations, there will be a certain number of unexpected high-cost healthcare expenses, but the larger the population, the more likely this number will be relatively stable, minus any external factors, from year to year. Thus, larger populations are much more amenable to reliable and accurate predictions for annual cost targets, for example.

When the number of members from a particular employer is too small to generate a reliable annual cost target (or for some other time period), the members of this particular employer may be combined, or pooled, with the members of other similarly small employers for the purpose of generating a preliminary cost target 234. This pooling of the members of two or more employers is intended to provide a large enough member population to allow for generation of a reliable cost target 236. In a particular embodiment, the server 102 is programmed to automatically combine members of employers where the member population of each employer individually is below a predetermined threshold level 232. With respect to the flowchart of FIG. 2, in some embodiments, the step 232 in FIG. 4 is performed after step 202. If the member population is above the threshold level, the method proceeds to step 204 and the method of FIG. 2 is unchanged. If the member population is below the threshold level, steps 234 and 236 are performed in place of step 204. Then the method proceeds to step 206 and then proceeds in accordance with FIG. 2.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of aligning costs in a healthcare system, the method comprising:
providing a networked computer server configured to receive, via the network, personal and health data for each of a plurality of members in the healthcare system;
aligning at least a portion of the plurality of members with a healthcare provider using a member alignment algorithm executed by the computer server;
calculating a preliminary cost target for total healthcare costs for a period of time using a cost target algorithm executed by the computer server, the preliminary cost target based on a baseline historical medical costs;
determining a contribution margin that includes both a fixed cost and a profit margin for the healthcare provider, and determining a variable cost for one or more healthcare services;
calculating fixed cost payments to the healthcare provider on a per-member-basis;
receiving claim data, via the network, for one or more claims made by the one or more of the plurality of members; and
calculating claim payments on a per-member-basis, the claim payments based on the variable costs associated with the claim for healthcare services.

2. The method of claim 1, further comprising tracking all fixed cost payments and all claim payments for the period of time to determine a revised healthcare cost, and comparing the revised healthcare cost to the preliminary cost target.

3. The method of claim 2, further comprising calculating a risk sharing component where any cost saving realized when the revised healthcare cost is less than the preliminary cost target, or any cost overage realized when the revised healthcare cost is greater than the preliminary cost target, is divided between the healthcare provider and a customer of the healthcare provider.

4. The method of claim 3, further comprising configuring the computer server to allow a user to adjust how the risk sharing component is divided.

5. The method of claim 2, wherein determining a revised healthcare cost comprises excluding some claim payments to get an adjusted revised healthcare cost.

6. The method of claim 5, wherein excluding some claim payments comprises excluding some claim payments above a certain threshold amount.

7. The method of claim 1, further comprising calculating an adjusted risk score for a member using a risk adjustment algorithm executed on the computer server, the adjusted risk score indicating a healthcare cost risk for the member as compared to a sample population.

8. The method of claim 7, further comprising calculating an adjusted cost target based on the adjusted risk score for each of the plurality of members.

9. The method of claim 1, wherein calculating the preliminary cost target for total healthcare costs using a cost target algorithm comprises excluding some healthcare costs from the historical baseline cost to get an adjusted baseline cost, and trending the adjusted baseline cost forward to cover a current period of time.

10. The method of claim 9, wherein excluding some healthcare costs comprises excluding some healthcare costs above a certain threshold amount.

11. The method of claim 1, further comprising pooling members of two or more employers before calculating the preliminary cost target for the pooled members.

12. A healthcare cost alignment system comprising:
a computer server configured to communicate, via a communications network, with one or more client computers in order to send and receive healthcare information over the communications network, the computer server configured to:
receive personal and healthcare data for each of a plurality of members in a healthcare system;
align each of the plurality of members with a healthcare provider based on a set of criteria for member alignment;
determine a preliminary cost target for total healthcare costs based on historical data and the personal and healthcare data, and a variable cost for one or more healthcare services;
receive, over the network, claim data for one or more claims made by one or more of the plurality of members; and
calculate a claim payment amount for each of the one or more claims based on variable costs associated with the claim at issue.

13. The healthcare cost alignment system of claim 12, wherein the computer server is further configured to track all fixed cost payments and all claim payments for the period of time to determine a revised healthcare cost, and compare the revised healthcare cost to the preliminary cost target.

14. The healthcare cost alignment system of claim 13, wherein the computer server is further configured to calculate a risk sharing component where any cost saving realized when the revised healthcare cost is less than the preliminary cost target, or any cost overage realized when the revised healthcare cost is greater than the preliminary cost target, is divided between the healthcare provider and a customer of the healthcare provider.

15. The healthcare cost alignment system of claim 14, wherein the computer server is further configured to allow a user to adjust how the risk sharing component is divided.

16. The healthcare cost alignment system of claim 13, wherein the computer server is further configured to determine a revised healthcare cost that excludes some claim payments in order to get an adjusted revised healthcare cost.

17. The healthcare cost alignment system of claim 16, wherein the computer server is further configured to exclude some claim payments above a certain threshold amount.

18. The healthcare cost alignment system of claim 12, wherein the computer server is further configured to calculate an adjusted risk score for a member using a risk adjustment algorithm executed on the computer server, the adjusted risk score indicating a healthcare cost risk for the member as compared to a sample population.

19. The healthcare cost alignment system of claim 18, wherein the computer server is further configured to calculate an adjusted cost target based on the adjusted risk score for each of the plurality of members.

20. The healthcare cost alignment system of claim 12, wherein the computer server is further configured to exclude some healthcare costs from the historical baseline cost to get an adjusted baseline cost, and configured to trend the adjusted baseline cost forward to cover a current period of time.

21. The healthcare cost alignment system of claim 20, wherein the computer server is further configured to exclude some healthcare costs above a certain threshold amount.

22. The healthcare cost alignment system of claim 12, wherein the computer server is further configured to pool members of two or more employers before calculating the preliminary cost target for the pooled members.

23. The healthcare cost alignment system of claim 22, wherein the computer server automatically pools the members of any employer with less than a threshold number of members.
